(19) United States
(12) Patent Application Publication
(10) Pub. No.: US 2007/0237837 A1
(43) Pub. Date: Oct. 11, 2007

(54) BIOCOMPATIBLE TEA TREE OIL COMPOSITIONS

(75) Inventors: Gregory Pipko, Katzrin (IL); Moshe Reuveni, Katzrin (IL); Dani Neifeld, Moshav Bney Yehuda (IL); Yaakov Spector, Srigim Leon (IL)

Correspondence Address: BROWDY AND NEIMARK, P.L.L.C.;624 NINTH STREET, NW, SUITE 300, WASHINGTON, DC, 20001-5303, US

(73) Assignee: BIOMOR ISRAEL LTD., Katzrin (IL)

(21) Appl. No.: 11/695,308

(22) Filed: Apr. 2, 2007

Related U.S. Application Data
(63) Continuation-in-part of application No. 10/511,755, filed on Oct. 19, 2004.
(63) Continuation-in-part of application No. 10/968,146, filed on Oct. 20, 2004, now abandoned.

(30) Foreign Application Priority Data
Sep. 4, 2002 (IL) 151594
Oct. 20, 2003 (IL) 158509

(51) Int. Cl. A61K 36/00; A61K 33/00

(52) U.S. Cl. 424/717; 424/195.18; 424/719; 424/725; 424/729; 424/736; 424/742; 424/745; 424/747; 424/750; 424/769; 424/770; 424/778

(57) ABSTRACT

A non-phytotoxic fungicidal composition comprising an alkali or ammonium salt of a high MW organic fatty acid forming an emulsion, and tea tree essential oil, terpinen-4-ol oil solubilized in the emulsion, and a method of producing the aforesaid non-phytotoxic biocide, including the steps of admixing alkali or ammonium salts with organic fatty acids so an emulsion is obtained, and admixing terpinen-4-ol oil in the emulsion until full solubilization is obtained.

This is a Continuation-In-Parts application of non-provisional applications, namely application U.S. Ser. No. 10/511,755 filed on Oct. 19, 2004; and U.S. Ser. No. 10/968,146 filed on Oct. 20, 2004.

### ABSTRACT

The present invention discloses non-phytotoxic biocompatible compositions, comprising tea tree oil (TTO) in stable water-in-oil (W/O) emulsion, method for its production and methods for treating soil pathogens, plant pathogens or pathogens in aquaculture.

### FIELD OF THE INVENTION

The present invention generally relates to preparations with tea tree oil specifically of the species Melaleuca alternifolia, (hereinafter TTO) characterized by biocompatible biocide composition containing TTO and to a method of its production.

### BACKGROUND OF THE INVENTION

TTO is an essential oil characterized by a broad-spectrum antiseptic activity and is a very effective biocide against bacteria, fungi and as an insect repellant. TTO is an essential oil distilled from the leaves of the paperbark tree species Melaleuca alternifolia. The tree is indigenous to the moist, sub-tropical coast of northeastern New South Wales and southeast Queensland in Australia, and has evolved its own natural defenses against disease and its own natural repellants against insects.

It is known that the major antiseptic active component of the TTO is the Terpinen-4-ol family. MSGC identified terpinen-4-ol (about 42%), a-terpineol (about 3%) and 1,8-cineole (about 2%, respectively, of tea tree oil) as the water soluble components of tea tree oil. The mode of action of TTO on cell target is to damage the pathogen's cell wall and membrane and subsequently to denature the cell constituents. The antiseptic actions of TTO are not impaired in the presence of blood, serum, pus, mucous discharge etc. An acquired immunity of microorganisms to many antibiotics and sulphonamide drugs does not occur with TTO.

Substantial microbiological testing of TTO has established in the literature typical inhibitory concentrations of the oil against a broad spectrum of microorganisms. Nevertheless, its sharp aromatic characteristics eliminate it is use AS IS in humans, animal and field-corps. Many formulations have been suggested in the art, few of them teach its use in emulsions. U.S. Pat. No. 6,464,989 to Dillon for example discloses tea tree oil emulsion formulations at a concentration greater than about 22% on a weight-by-weight basis. The emulsion also contains an emulsifier, wheat germ oil, cocoa butter, beeswax, ozokerite wax, pentaerythritol ether, vitamin E acetate, vitamin A, and vitamin D3. The emulsifier is selected from stearic acid, glyceryl stearate, polyethylene glycol 100 stearate, steareth-21, and steareth-2. This cosmetic formulation is especially adapted to be topically administrated on humans skin, is evidently not suitable for any agricultural utilization.

It is acknowledged in this respect that some TTO-containing compositions suggested in the art are targeted for herbicidal purposes. The highly phyto-toxic nature of the TTO is widely used in many commercially available herbicide products. U.S. Pat. No. 5,998,335 to Selga et al. presents one approach for a “knock-down” herbicidal composition effective against mature weeds having, as its sole active ingredient, a combination of (a) pine oil and (b) tea tree oil or eucalyptus oil. As set forth above, the TTO comprises highly volatile and effective ingredients, which kill vegetable cells, microorganisms, insects and other pathogens.

While few effective TTO-containing emulsion biocides have been published in the literature, their harmful side effects were not considered at all. Thus for example, U.S. Pat. No. 5,610,189 to Whiteley discloses a disinfecting composition comprising stable aqueous solutions of (a) a blend of biocide active terpenes from TTO; (b) one or more biocide active surfactants; (c) one or more proton donor type biocides; and (d) a salt of mono, di- or trihydroxy aliphatic or aromatic acid; and U.S. Pat. No. 6,197,305 to Friedman et al. discloses composition for oral hygiene for treating a fungal infection, comprising: a mixture of herbal extracts; (b) a mixture of essential oils such as TTO; and a pharmaceutical carrier; wherein said herbal extracts are each present in an amount of from about 1% to about 10% by weight, and each essential oil is present in an amount of from about 0.2% to about 2.0% by weight.

JP Pat. No. 2,145,502 to Watanabe et al. teaches an agent for controlling crop diseases by means of TTO adapted to the form of a dust or a granular preparation. Those particles in the solid phase are introduced to the object to be treated by means of cloth or paper so that the powder flies off. This approach suffers several drawbacks, i.e., the TTO-containing power is characterized by only meager wetting properties, its delivery to the crops and the pathogens is very much limited, it is suitable only for a narrow range of applications, i.e., in-house gardening and especially the respectively expensive plants accommodated in flowerpots.

Methyl bromide is a colorless neurotoxic gas commonly utilized as a soil biocide, for controlling nematodes, weeds and fungi, primarily for tomatoes, ornamentals, tobacco, peppers, strawberries and forest seedlings, however now widely banned for use. Soil disinfection with methyl bromide takes place only prior to planting or seeding. No use is currently made post seeding or planning. Increasing limitations are being imposed on the use of this material due to its being a significant ozone layer damaging gas. Attempts to develop alternatives to eliminate or reduce its use are taking place with limited results.

Various alternative disinfestation methods were presented in the art, such as sterilization at greater than 100° C. or pasteurization at 70° C. that eliminate many pathogenic fungi and their specific survival forms. Chemical treatments are selected from total disinfectants and limited biocides or biostatic activity agents.

The Montreal Protocol (2001) hinders the use of compounds that have a detrimental effect on the ozone layer, including the methyl bromide. Many alternatives were suggested, including (i) Phosphine, which is highly toxic to organisms undergoing oxidative respiration; and (ii) 1,3-Dichloropropene which causes irritation at the point of contact. It causes nausea, vomiting, irritation of the skin, eyes, and throat; breathing difficulties, headache, and fatigue. Ingestion of the composition causes death. Animal studies have reported damage to the stomach lining, lung congestion, difficulty in walking, and effects on the liver and kidneys from ingesting high levels of 1,3-dichloropropene. (iii) 3,5-dimethyl-1,3,5-thiadiazinane-2-thione is a potential ground water contaminant which indicates high toxicity in the given toxicological category. Thus, the cost-effectiveness of those alternatives in treating nematodes, weeds and fungal pathogens is not yet shown.

Hence for example, melon cultivation was introduced to Guatemala as a cash crop in the early 1970s. The climatic conditions in the northeastern region of Zacapa allow the production of high quality fruits. Some 50% of the cultivated area in the region is being treated with methyl bromide at a rate of 250-300 kg/Ha. The diminishing quotas are applied on highly infested plots. Soil mulching is applied annually on an area of 2,000 Ha.

As the cultivated area increased over 20-30 years of successive cropping, soil-borne pests appeared, especially Fusarium spp., Root knot nematodes of the Meloidogyne species, Acidovorax avenae in seed producing plots, were followed by heat-tolerant Monosporascus cannonballus and the recently diagnosed melon necrotic spot virus (MNSV) transmitted by Olpidium bornovarus. The main weed problem is Cyperus rotundus. Generally accepted methyl bromide alternatives failed to deliver consistent results under the conditions of Guatemala. Hence, 1,3-Dichloropropene (namely commercially available Telone ™) appeared to be corrosive to drip irrigation systems. Solarization is less effective due to heat-tolerant fungus. Little experimentation was done with 3,5-dimethyl-1,3,5-thiadiazinane-2-thione (namely commercially available Dazomet ™) and the results obtained with sodium methyldithiocarbamate (such as commercially available Metam Sodium) are less satisfactory for the control of the sudden wilt.

All the chemical soil disinfection methods used today are based on chemical synthetic fumigants.

Provision of pesticides through irrigation, and in all cases synthetic and target specific pesticides, is known in the art. Systemic and synthetic fungicides are used in certain crops wherein the systemic fungicides are provided through irrigation and absorbed into the plant through its water absorption system and protect it against pathogenic fungi.

No viable broad spectrum solution has been reported which is an organic product whose active ingredients are all from natural sources, and which is a broad spectrum product, and useful both as soil disinfectant prior to seeding or planting AND during the plant growth.

The soil resident Aphid Eriosoma lanigerum is a pest very difficult to eradicate. The pathogen attacks the bark of fruit trees. The standard treatments are based on organic phosphor compositions which are toxic to worm-blooded animals and degrade very slowly in the field. Another negative impact of these materials is its toxic effect on the only natural predator, Aphelinus mali, for which the composition in this invention is safe. No natural, environmental friendly pesticide is currently available for treating this pest.

Hence for example, melon cultivation was introduced to Guatemala as a cash crop in the early 1 970s. The climatic conditions in the northeastern region of Zacapa allow the production of high quality fruits. Some 50% of the cultivated area in the region is being treated with methyl bromide at a rate of 250-300 kg/Ha. The diminishing quotas are applied on highly infested plots. Soil mulching is being applied annually on an area of 2,000 Ha. As the cultivated area increased over 20-30 years of successive cropping, soil-borne pests appeared especially Fusarium spp., Root knot nematodes of the Meloidogyne species, Acidovorax avenae in seed producing plots, to be followed by heat-tolerant Monosporascus cannonballus and the recently diagnosed melon necrotic spot virus (MNSV) transmitted by Olpidium bornovarus. Generally accepted methyl bromide alternatives failed to deliver consistent results under the conditions of Guatemala. Hence, 1,3-Dichloropropene (namely commercially available Telone ™ appeared to be corrosive to drip irrigation systems. Solarization is less effective due to heat-tolerant fungus. Little experimentation was done with 3,5-dimethyl-1,3,5-thiadiazinane-2-thione (namely commercially available Dazomet ™) and the results obtained with sodium methyldithiocarbamate (such as commercially available Metam Sodium) are less satisfactory for the control of the sudden wilt.

Diluted solutions of tea tree oil are often used as a remedy to treat bacterial and fungal infection in pet fish. Common brand names are Pimafix ™ (Pimenta racemosa oil, 1%) Melafix ™ (cajeput oil, 1%) and Bettafix ™ (cajeput oil, 0.2%). It is most commonly used to promote fin and tissue re-growth but is also claimed to be effective in preventing other conditions such as fin rot or velvet. Effective treatment of acute fungal diseases is not yet disclosed. The remedy is used mostly on Betta fish but can also be used with other pet fish. Melafix and Bettafix are based on cajeput oil, obtainable from leaves of the East Indian tree Melaleuca cajuputi, and M. leucadendron.

It has been shown that tea tree oil inhibits certain fungi (See for example Australian Journal of Experimental Agriculture 39:1, 86-81, 1999). The treatment was satisfactory as it killed the fungi to a large extent, and mainly fungi that attack human, while in plants it caused phyto-toxicity to attacked plants.

An invention of a biocompatible, especially non-phytotoxic, aquaculture-friendly and ozone-safe biocides, would meet an ever growing industrial need.

### SUMMARY OF THE INVENTION

It is thus the core of the invention to provide cost-effective biocompatible compositions comprising tea tree oil (TTO as defined above) in stable water-in-oil (W/O) emulsion of alkali or ammonium salts of organic fatty acid wherein said emulsion is also stable when converted into an oil-in-water emulsion.

It is one object of the invention wherein the biocompatible composition defined above is provided useful for plant protection, excluding roots and seeds, from their pathogens, wherein said TTO-based emulsion is non-phytotoxic.

It is in the scope of the invention wherein the organic fatty acids comprising linear or branched alkyl chains of 6<C<22, and especially linear or branched alkyl chains of 12<C<18.

It is also in the scope of the invention wherein at least a portion of the organic fatty acids is saturated fatty acid selected among lauric acid, myristic acid, palmitic acid, stearicacid, arahinoic acid, behenic acid, lignocerinic acid or any mixture thereof.

It is also in the scope of the invention wherein at least a portion of the organic fatty acids is unsaturated fatty acids selected among decenoic acid, dodecenoic acid, palamitinoleic acid, oleic acid, lonoleic acid, undecelenic acid, sorbic acid, recinoleic acid or any mixture thereof.

It is also in the scope of the invention wherein at least a portion of the organic fatty acids is selected among tall oil acids, tall oil fatty acid (TOFA), naftenic acids, rosin acids and any mixture thereof. It is acknowledged in this respect that Tall oil may consist of TOFA and/or resins thereof, and/or TOFA per se.

It is also in the scope of the invention wherein the aforesaid biocompatible compositions additionally comprising etheric oil. The etheric oil is possibly, yet not exclusively selected for a group consisting inter alia lavender (Lavandula angustifolia) oil, pine (Pinus sylvestris) oil, manuka (Leptospermum scoparium) oil, kanuca (Kunzea ericoides) oil, eucalyptus (Eucalyptus globulus) oil, bergamot (Citrus bergamia) oil, clove (Eugenia caryaphylata) oil, lemon (Citrus limoneum) oil, lemon grass (Cymbpogon citratus) oil, rosemary (Rosmarinus officialis) oil, geranium (Pelargonium graveoleus) oil, mint oil, which refers hereinafter either to mint oil or to any other composition containing high levels of menthol and/or menthene or any mixture thereof.

Compositions containing high levels of menthol and/or menthene include but are not limited to peppermint oil, cornmint oil, spearmint oil, horsemint oil, round leaf mint oil, oil of hyssop, Japanese mint oil, European pennyroyal, and American pennyroyal.

It is also in the scope of the invention wherein a water-in-oil non-phytotoxic emulsion is provided. The concentration of TTO is about 10% to about 70% (weight percent), based on the total weight of said emulsion, and, when converted into an oil-in-water emulsion the concentration of said TTO is about 0.01% to about 1.5% (weight percent), based on the total weight of the emulsion.

It is also in the scope of the invention to disclose a water-in-oil aquaculture-safe emulsion as defined above, wherein the oil-in-water emulsion comprising TTO is a concentration which varies from about 0.00015% to about 1.5% (weight percent), based on the total weight of the emulsion.

It is also in the scope of the invention wherein the concentration of the etheric oil (admixed to the emulsion) is between 0.01% by weight to 50% by weight.

It is also in the scope of the invention wherein the concentration of terpinen-4-ol in the TTO is in the range of 20% by weight to 50% by weight.

It is also in the scope of the invention wherein the TTO comprising inter alia compounds selected from terpinenes, cymenes, pinenes, terpineols, cineole, sesquiterpenes, sequiterpene alcohols or any mixture thereof.

It is also in the scope of the invention wherein the alkali and ammonium salts comprising at least one compound selected from sodium, potassium and/or ammonium hydroxides, carbonates, bicarbonates or any mixture thereof.

It is also in the scope of the invention wherein the aforesaid emulsion comprising surfactants selected from a group consisting of cationic, anionic or zwitterionic, non-ionic (especially Tween 20™ or the like) or a mixture thereof.

It is also in the scope of the invention wherein the aforesaid emulsion additionally comprising antioxidants, especially tocopherol, retinol, ascorbates or any mixture thereof.

It is also in the scope of the invention wherein the emulsion as defined in any of the above is characterized by a non-phytotoxic broad spectrum disinfectant or repellant activities selected from kanacidic, insectecidic, arachnidacidic, antibiotic, fungicidic, nematocidic, bacteriocidic activities or any mixture thereof.

It is also in the scope of the invention wherein the aforesaid emulsion additionally comprises of tetracyclo-quinolizindine alkaloids, natural pyrethrines (and products of chrysanthemum Daisy, Tanacetum cinerariarfolium), matrine, oxymatrine, alkaloids or any mixture thereof.

It is also in the scope of the invention wherein the aforesaid alkaloids are extracted from a group consisting of chrysanthemum Daisy, Tanacetum cinerariarfolium, Sophora japonica, Sophora subprostrata, Sophora alopecuroides or any combination thereof.

It is also in the scope of the invention wherein the aforesaid TTO comprising at least one oil selected from a group consisting of terpinen-4-ol, a-terpineol, 1,8-cineole, α and γ terpinen or a combination thereof.

It is another object of the invention wherein biocompatible ozone-safe biocides comprising soil-treating disinfectants as defined above is obtained.

It is also in the scope of the invention wherein the aforesaid soil-treating disinfectants are non-phytotoxic soil-treating disinfectants especially useful for targeting plant pathogens. Those soil-treating disinfectants are especially useful when applied to the soil pre-seeding, pre-seedlings and/or during plants life time.

It is also in the scope of the invention wherein the aforesaid soil-treating disinfectants are useful for treating bacteria, nematodes and fungi.

It is another object of the invention wherein a biocompatible composition as defined in any of the above is provided useful for the management of fungi pathogens in aquaculture, especially in fish farming, and particularly in treating Saprolegnia infections.

It is another object of the invention to disclose a method of producing a biocompatible biocide composition.

It is another object of the invention to disclose a method of producing a biocompatible biocide composition. This method comprises of steps selected inter alia from (a) admixing water-immiscible ingredients, optionally, admixing up to 100% of TTO; (b) admixing water-miscible ingredients, especially alkali or ammonium salts such that a stable water-in-oil emulsion is obtained; and, (c) optionally, admixing the remaining TTO in said emulsion.

It is also in the scope of the invention wherein another embodiment of the method comprises of steps as follows: producing a water-in-oil emulsion; the concentration of said tea tree oil is about 10% to about 70% (weight percent), based on the total weight of said emulsion; and, converting said water-in-oil emulsion into an oil-in-water emulsion, the concentration of said tea tree oil is about 0.01% to about 1.5% (weight percent), based on the total weight of said emulsion.

It is also in the scope of the invention wherein another embodiment of the method which especially adapted for the production of non-phytotoxic TTO-based plant-protecting emulsion.

It is also in the scope of the invention wherein another embodiment of the method comprising a step or steps of admixing water-immiscible ingredients that comprises of organic fatty acids. The fatty acids are preferably, yet not exclusively, selected from a group consisting of linear or branched alkyl chains of 6<C<22; especially wherein at least a portion of the organic fatty acid is saturated fatty acid selected among lauric acid, myristic acid, palmitic acid, stearic acid, arahinoic acid, behenic acid, lignocerinic acid or any mixture thereof, especially wherein at least a portion of the organic fatty acid is unsaturated fatty acids selected among decenoic acid, dodecenoic acid, palamitinoleic acid, oleic acid, lonoleic acid, undecelenic acid, sorbic acid, recinoleic acid or any mixture thereof; especially wherein at least a portion of the organic fatty acid is selected among tall oil acids, tall oil fatty acid, naftenic acids, rosin acids and any mixture thereof.

It is also in the scope of the invention wherein another embodiment of the method comprising step or steps of admixing water-immiscible ingredients that comprises of at least one etheric oil. The etheric oil is selected in a non-limiting manner for a group consisting of lavender (Lavandula angustifolia) oil, pine (Pinus sylvestris) oil, manuka (Leptospermum scoparium) oil, kanuca (Kunzea ericoides) oil, eucalyptus (Eucalyptus globulus) oil, bergamot (Citrus bergamia) oil, clove (Eugenia caryaphylata) oil, lemon (Citrus limoneum) oil, lemon grass (Cymbpogon citratus) oil, rosemary (Rosmarinus officialis) oil, geranium (Pelargonium graveoleus) oil, mint oil or any mixture thereof, especially wherein the total concentration of said at lest one etheric oil (admixed to the emulsion) is between 0.01% by weight to 50% by weight or especially wherein the terpinen-4-ol concentration in the TTO is in the range of 20% by weight to 50% by weight.

It is also in the scope of the invention wherein the method as defined in any of the above additionally comprises step or steps of admixing surfactants. The surfactants are selected in a non-limiting manner from a group consisting of cationic, anionic or zwitterionic, non-ionic (especially commercially available polyoxyethylene (20, 80 etc) sorbitan monolaurate, e.g., Tween 20™) or any mixture thereof

It is also in the scope of the invention wherein the method as defined in any of the above additionally comprises step or steps of admixing antioxidants, especially tocopherol, retinol, ascorbates or any mixture thereof.

It is also in the scope of the invention wherein the method as defined in any of the above wherein the emulsion is characterized by a non-phytotoxic broad spectrum disinfective activity and/or insect repellant activity selected, especially wherein this disinfective activity is selected in a non-limiting manner from a group consisting of kanacidic, insectecidic, arachnidacidic, antibiotic, fungicidic, nematocidic, bacteriocidic activities or any combination thereof.

It is also in the scope of the invention wherein the method as defined in any of the above additionally comprises of a step or steps of admixing compositions selected in a non-limiting manner from a group consisting of tetracyclo-quinolizindine alkaloids, natural pyrethrines, products of chrysanthemum Daisy, Tanacetum cinerariarfolium, matrine, oxymatrine, alkaloids or any mixture thereof, especially wherein the alkaloids are extracted from the group consisting of chrysanthemum Daisy, Tanacetum cinerariarfolium, Sophora japonica, Sophora subprostrata, Sophora alopecuroides or any combination thereof.

It is also in the scope of the invention wherein the method as defined in any of the above additionally comprises step or steps if admixing TTO which comprises inter alia at least one oil selected from a group consisting of terpinen-4-ol, a-terpineol, 1,8-cineole, α and γ terpinen or a mixture thereof.

It is also in the scope of the invention wherein the method as defined in any of the above is adapted for targeting plant pathogens by non-phytotoxic ozone-safe plant biocides. This method comprises steps selected from (a) obtaining TTO-based soil-disinfecting emulsions as defined in any of the above; and (b) applying said emulsions to said soil and/or to said plant in an effective amount for the purpose whilst said application is provided prior to either planting or seeding. It is also in the scope of the invention wherein the method as defined in any of the above is adapted for treating bacteria, nematodes or fungi.

It is also in the scope of the invention wherein the method as defined in any of the above is adapted for treating fungi pathogens in aquaculture, especially in fish farming, and particularly in treating Saprolegnia infections by biocompatible biocides. This method comprises of steps selected inter alia from (a) obtaining TTO-based aquaculture-disinfecting emulsions according to the method defined in claim 25; and (b), introducing said emulsions to the water an effective amount for the purpose until pathogens are treated.

### DETAILED DESCRIPTION OF THE INVENTION

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide biocide compositions containing TTO (hereinafter ‘biocide composition’), and more specifically to provide an effective emulsion containing etheric components obtained biocompatible biocide compositions.

This biocide product comprising TTO and a water emulsion; wherein the emulsifier is a water solution of a reaction product of a high molecular weight organic fatty acid and an alkali or ammonium compound.

The term ‘tea tree’ (i.e., TT) is especially referring hereinafter to Melaleuca alternifolia known in the common name “tea tree.” More generally, the term is referring to any of the laurel tree family, unusual variety indigenous to the east coast of Australia. The term ‘tea tree’ (i.e., TT) is more generally referring hereinafter to any tree of the tea tree family and to other Australian trees or plants of other locations provided for useful essential oil as such as those obtained from tree of the family of the Eucalyptus, particularly Eucalyptus citriodora, Eucalyptus globules, Eucalyptus radiata; and other plants, such as niaouli oils (Melaleuca viridiflora)—Australian Niaouli, also known as broad-leaved paperbark; Australian Blue Cypress; Kunzea ambigua, also known as “White cloud” or “White kunzea”; Lavandula angustifolia (e.g., Bridestowe), known also as Tasmanian Lavender; Western Australian Sandalwood; White (e.g., Jade) Cypress—Essential oil of Callitris columellaris/glaucophylla; Citrus bergamia; Mentha piperita; Rosmarinus officinalis; Pine oils; Essential Oil Blends, such a blends of Melaleuca alternifolia, Melaleuca quinquenervia nerolidol/linalool, and Callitris intratropica etc.

The term ‘tea tree oil’ (i.e., TTO) is generally referring hereinafter to any water-miscible and/or water-immiscible ingredient or product obtained from Melaluca Alternafolia. Particularly, the term TTO relates to mixtures comprises inter alia terpinen-4-ol, terpinenes, cymenes, pinenes, terpineols, cineole, sesquiterpenes, and sequiterpene alcohols. The term TTO is also referring to any naturally obtained or chemically synthesized of purified composition or comprises of terpinen-4-ol oils, 28-48%; γ-terpinene, 10-28%, α-terpinene, 2.7-13%; 1,8,-cineole, 0.1-16.5%, and various terpenes, 1-25% selected yet not limited to α-pinene, limonene, ρ-cymene and terpinolene. This term is also referring to Phlai Oil, e.g., an essential oil obtained from rhizome Zingiber cassumunae.

The term ‘emulsion’ is referring hereinafter to any water in oil (W/O); oil in water (O/W); W/O/W and/or O/W/O phases comprising the TTO inside, outside or at the surface of aggregates, vesicles, micelles, reversed micelles, nano-emulsions, micro-emulsion, liposomes or in any combination thereof.

The term ‘emulsifier’ is referring hereinafter to any material or molecule provided as a polymer, oligomer or monomer and is nonionic, anionic or cationic detergent and/or surfactant. The emulsifier is preferably comprises of both lypophilic and hydrophilic portions, such as in saturated or non-saturated long chain alkyl comprising at least one polar or charged atom. It is e.g., in the scope of the invention wherein commercially available Tween 20™ emulsifies are used.

The terms ‘biocide’ is specifically referring hereinafter to consisting biocompatible non-phytotoxic biocides selected from yet not limited to miticides, insecticides, arcanidacides, algaecides, bactericides, fungicides or any combination thereof.

For purposes of the present invention, the term “an effective amount for the purpose” is defined as the amount of TTO-based composition which when added to the soil and will control the deleterious organisms, yet will not exhibit phyto-toxicity to the plants, because of the specific methods and timing of the addition.

The term ‘soil’ refers to any soil capable of growing a food or an ornamental crop such as strawberries, almonds, grapes, ornamental flowers, tobacco, tomatoes, watermelon, grass sod, apples, peanuts, lettuce, soybeans, onions, peaches, sugar cane, wheat, cherries, and other field crops and ornamental species.

Typical devices for applying effective amount of TTO-based soil-treating disinfectants compositions to the soil include a gravity flow applicator, e.g., chisel, tooth or shank type applicators; commercially available sprayers, atomizers, aerators, blowguns, blowpipes; pulverizes or the like are also provided as useful applicators. Irrigating means, such as drip emitters, micro sprayers, emitter tubing, misters and the like are useful applicators. Other methods of delivery useful in the method of the present invention include encapsulation, micro-encapsulation or any commercially available techniques of controlled release of flowing matter.

The non-phytotoxic biocide compositions according to the present invention are proved to be environmentally friendly: its principal constituent; TTO and/or terpen-4-ol, does not pollute. The compositions are extremely effective biocide and characterized by a wide spectrum, no-gap biocide: sanitizing disinfectant effective against—bacteria, viruses, fungi and algae, a simple ‘one-stop’ approach. Those products are proved to destroy biofilm and have long term effectiveness. It is simple application, and simple to monitor: product concentrations can be easily and accurately measured and know to be safe: in its diluted state it does not cause irritation to the skin, eyes, and mucous membranes, nor is it toxic or have any known carcinogenic or mutagenic effects.

This biocide composition consists in a biocide emulsion comprising TTO and a water emulsion in which the emulsifier is provided in a non-limiting manner by a water solution of a reaction product of a high molecular weight organic fatty acid and an alkali or ammonium compound.

Additionally or alternatively, the emulsions are produced by (a) admixing water-immiscible compositions (e.g., etheric oils, organic fatty acids, tall oil etc); and then (b) admixing water-miscible compositions (e.g., alkali or ammonium salts etc), such that a stable W/O is obtained. Those emulsions are stable, e.g., no phase separation or chemical instability was obtained after 2 years storage at ambient temperature. Hence for example, TTO or its constituents are admixed to the water-immiscible compositions in step (a). Alternatively, a predefined measure of the total TTO or its constituents is admixed to the water-immiscible compositions in step (a), whilst the remaining portion is admixed with the water-miscible composition at step (b). It is acknowledged in this respect that admixing is provided e.g., by high rate shearing homogenation, shaking, slow and gentle stirring or any combination thereof.

It is another embodiment of the present invention wherein the aforementioned water immiscible compositions is selected inter alia from high molecular weight fatty acids, fatty acids or a mixture of said acids, wherein those acids are saturated, unsaturated or a combination of the two, one or all referred hereto in the short term ‘high molecular weight fatty acids’.

It is another embodiment of the present invention wherein the aforementioned high molecular weight fatty acid comprising linear or branched alkyl chains of C>6 to C<22, especially the range from C>12 to C<18 atoms per molecule.

It is in the scope of the present invention wherein the aforesaid high weight fatty acids are selected in a non-limiting manner from at least one of the following groups:
i. tall oil fatty acids, naftenic acids, rosin acid or any combination thereof;
ii. saturated fatty acids selected from the group of lauric acid, myristic acid, palmitic acid, stearic acid, arahinoic acid, behenic acid, lignocerinic acid or any combination thereof;
iii. unsaturated fatty acids selected from the group of decenoic acid, dodecenoic acid, palamitinoleic acid, oleic acid, lonoleic acid, undecelenic acid, sorbic acid, recinoleic acid or any combination of thereof.

According to yet another embodiment of the present invention, those acids are treated with water miscible compositions, selected in a non-limiting manner from alkali hydroxides, carbonates, bicarbonates or any combination thereof to obtain a salt. Additionally or alternatively, the hereto-defined acids are admixed with sodium, potassium or ammonium compounds, e.g., hydroxides, carbonates, bicarbonates or any combination thereof to obtain a salt.

According to yet another embodiment of the present invention, a water-in-oil emulsion is provided, wherein the TTO concentration is about 10% to about 70% (weight percent), based on the total weight of the emulsion, and, when converted into an oil-in-water emulsion the TTO concentration is about 0.01% to about 1.5% (weight percent), based on the total weight of said emulsion.

According to yet another embodiment of the present invention, a water-in-oil emulsion is provided, wherein the TTO concentration is about 10% to about 70% (weight percent), based on the total weight of the emulsion, and, when converted into an oil-in-water emulsion the TTO concentration is about 0.00015% to about 1.5% (weight percent), based on the total weight of said emulsion.

According to yet another embodiment of the present invention, the emulsion additionally comprises of etheric oil. Hence, an oil-in-water TTO-based emulsion comprises etheric oil in a concentration ranges of about 0.001% to about 5%, especially 0.1% to 2% (weight percent), based on the total weight of this O/W emulsion.

Freshly prepared salts solution in water give good emulsification of TTO in a wide concentration range. However, it is possible to use industrially prepared alkali salts of organic acid in powder or in granulated form to dissolve the salt obtained in hot water and to use the received solutions for the emulsification of the TTO.

The TTO containing biocides composition according to the present invention are useful for treating the pathogens located in the flowers, fruits, leaves, tubers, tubers, soil, etc. without alarming the plant itself.

It is also in the scope of the present invention to use the aforesaid TTO containing biocides useful for the management of pathogens in fish, especially in treating Saprolegnia infections. Moreover, said TTO compositions are useful as malachite green replacements.

In order to understand the invention and to see how it may be implemented in practice, a plurality of preferred embodiments will now be described, by way of non-limiting example only, with reference to the following examples, wherein all percentages are denoted for weight percents.

### EXAMPLE 1

300 g of naftenic acid are mixed with 160 g of 1 25% solution of NaOH in water for 60 minutes at 70° C. 316 g TTO is admixed to the reaction product obtained by a means of a contentious stirring until full homogenization is obtained. From the composition obtained, which contains 50% TTO, a stable TTO O/W emulsion is prepared by contentiously admixing of water, in the manner an emulsion comprising from 0.001% to 49.9% of oil.

### EXAMPLE 2

Into a 25% water solution of 300 g KHCO₃, some 400 g of melted stearic acid is admixed at 75° C. for 30 min. Subsequently; a mixture of 500 g of TTO and 200 g of lavender oil is admixed to the alkali admixture until a full homogenization is obtained. From the composition obtained, a stable TTO O/W emulsion comprising from 0.001% to 49.9% of etheric oil is obtained.

### EXAMPLE 3

30 g of Na₂CO₃ were dissolved in 100 g of water at 50° C. for 30 min. This solution was admixed with 120 g of tall oil acid, comprising 25% of rosin acid for additional 30 min. The obtained mixture was dissolved in 500 g of TTO until a homogenized solution is obtained. A plurality of stable TTO-containing emulsions was subsequently obtained.

### EXAMPLE 4

280 g of oleic acid was admixed with 85 g of a 20% ammonia solution at 60° C. 400 g of TTO was admixed until a homogenized solution was obtained.

### EXAMPLE 5

30 g of Na₂CO₃ were dissolved in 100 g of water at 50° C. for 30 min. This solution was admixed with 120 g of tall oil acid, comprising 25% of rosin acid for additional 30 min. The obtained mixture was dissolved in 250 g of TTO and 250 g of pine oil until a homogenized solution is obtained. A plurality of stable TTO-containing emulsions was subsequently obtained.

TABLE 1
An average effect of various biocides comprising TTO-containing
emulsions. Colonies of S. scabies (cfu per gram) were counted
under the microscope wherein average number of triplicates
of ten infected potato tubers is hereto present.

| | Bacteria | Inhibition |
|---|---|---|
| Treatment | cfu/gr | % |
| Control | 241.6 | — |
| 0.5% | 10.6 | 96 |
| 2.0% | 12.3 | 95 |

### EXAMPLE 6

The effect of the compositions defined above was tested in systems comprising aphids of various types. For example, those TTO containing compositions according to the present invention (i.e., TTO Composition A and B) where provided most effective in treating melon plants contaminated by green aphids. The melon plants were not affected or attacked by the said treatment.

TABLE 2
An average effect of various biocides comprising TTO-containing
emulsions on treating melon plants contaminated by green aphids

| | Aphids | Aphids | Aphids | Aphids |
|---|---|---|---|---|
| | No. | No. | No. | No. |
| Treatment | T₀ | T_{1 day} | T_{3 days} | T_{7 days} |
| Control | 156 | 161 | 103 | 98 |
| TTO Composition A | 197 | 52 | 22 | 13 |
| TTO Composition B | 274 | 20 | 5 | 3 |

### EXAMPLE 7

The effect of the compositions defined above was tested in systems comprising mites of various types. For example, those TTO containing compositions according to the present invention (i.e., TTO Composition A and B) where provided most effective in treating cucumbers, pepper and pumpkin plants contaminated by mites. All the aforementioned plants were not affected or attacked by the said treatment. The treatment included spraying the said compositions at the beginning of the experiments and after three consecutive days.

TABLE 3
An average effect of various biocides comprising
TTO-containing emulsions on treating cucumbers, pepper
and pumpkin plants contaminated by mites.

| | Mites No. | Mites No. | Mites No. |
|---|---|---|---|
| Treatment | T₀ | T*_{3 days} | T*_{5 days} |
| Control | 10 | 8 | 8 |
| TTO Composition A 1.0% | 10 | 2 | 0 |
| TTO Composition B 0.5% | 10 | 0 | 0 |

*Number of mites counted three and five days post treatment

### EXAMPLE 8

The effect of the compositions defined above was tested in systems comprising larva of Lepidifetera and/or Pectinophora Gossyiella Saunders. For example, those TTO containing compositions according to the present invention where provided most effective in treating cotton plants contaminated by this larva, especially by avoiding penetration of the larva into the plant, whereat control corps that was not initially sprayed by the TTO-compositions was significantly infected by the larva. Moreover, the cotton plants were not affected or attacked by the said treatment.

The present invention also provides a simple and novel method for producing the non-phytotoxic biocide as defined in any of the above. The method is essentially comprise two steps, yet other sub-reactions are possible: (A) admixing alkali or ammonium salts with organic fatty acids so an emulsion is obtained; and subsequently (B) admixing terpinen-4-ol oil in said emulsion until full solubilization is obtained. The mixing is provided by either stirring the solutions in a magnetic or mechanical stirrer or by means of any commercially available mechanical or ultrasonic homogenizer. The mixing is provided in a board spectrum of temperature, e.g., ambient temperature and/or at 25 to about 75° C. The mixing time is from 2 minutes to about 30 min., depend on the mixing velocity, admixed volume, admixing means, admixed compositions and temperature.

### EXAMPLE 9

The effect of TTO-containing biocompatible biocides for the management of pathogens in fish was studied. More specifically, the susceptibility of Saprolegnia to this novel disinfectant and antifungal agent was tested in both a both agar-dilution test, and apply the known NCCLS micro- and macro-broth dilution tests known in the art of medical mycology for the susceptibility tests.
Composition A: 10,000 ppm induced total inhibition; 1,000 ppm induced 80% inhibition in comparison to control; 100 ppm induces significant damage to the hyphae, and about 50% inhibition; 10 ppm were non effective at all.
Composition B: 1,000 ppm induced total inhibition; 100 ppm induced 80% inhibition; and 10 ppm induced 50%; 1 ppm was non-effective.

### EXAMPLE 10

The effect of the compositions defined above was tested for proving the effectively of aforesaid TTO-composition against Helminthosporium solani; Rhizoctonia solani, and Sterptomyces scabies in potatoes. Infected potato tubers were immersed in said compositions (0.5, 1.0 and 2.0%) for two minutes:

TABLE 4
An average disinfecting effect of various biocides comprising
TTO-containing emulsions on treating potato tubers contaminated
by various bacteria and fungi

| | Sterptomyces | Helminthosporium | Rhizoctonia |
|---|---|---|---|
| System | scabies | solani | solani |
| Control | 0 | 0 | 0 |
| 0.5% | 57 | 7.4 | 100 |
| 1.0% | 30 | 28.4 | 100 |
| 2.0 | 54 | 42.9 | 100 |

* Percent inhibition as related to control

### EXAMPLE 11

The effect of the TTO-compositions defined above was tested to prove a selective biological control of fungal and bacterial plant pathogens such that the mortality of biological control agent (namely Orius laeuvigatus) is respectively low. Hence, applying a direct spraying of the aforesaid TTO-emulsion (1%) on Orius laeuvigatus on pepper leaves according to the IOBC regulations was found harmless (mortality is lower than 30%).

### EXAMPLE 12

The effect of various TTO containing compositions based on the above was tested on representative list of plant pathogens, e.g., Powdery mildow (Leveillula taurica), Early blight (Alternaria solani), Powdery meldow (Odium spp.) and Powdery Mildow (Uncinula necator). No phyto-toxicity was detected on either of the neither compositions nor concentrations used.

TABLE 5
An average disinfecting effect of various biocides
comprising TTO-containing emulsions on treating plants
contaminated by various fungi, including various fungi
commonly defined as Powdery mildow (PM).

| | Plant | | | |
|---|---|---|---|---|
| | Pepper | Tarragon | Grapevines | Potatoes |
| | Pathogen | | | |
| | PM, | | | Early blight |
| | Leveillula | PM, Odium | PM, Uncinula | Altenraria |
| | taurica | spp | necator | solani |
| Efficacy | Infected | Infected | Infected | Infected |
| parameter | leaf area | shoots area | cluster area | shoots area |
| Control | 0 | 0 | 0 | 0 |
| 0.25% | 62.2 | na | 93.9 | 65.5 |
| 0.5% | 73 | 96.7 | 84.2 | 73.7 |
| 1.0 | 86.5 | 97.4 | 98.5 | na |

### EXAMPLE 13

The effect of a composition based on the above containing TTO (66%, weight percent) was tested against soil pathogens in a melon field, as an alternative to replace Methyl Bromide. Efficacy was tested against phyto-pathogenic fungi (Fusarium spp.) and nematodes (Meloidogyne, Aphelenchus, Hoplolimus and Rotylenchulus).

A field (0.3 hectare) was selected to run the experiment. From that field, soil and weed roots samples were taken for analysis. The field was drip irrigated by a single pump. The treatment rate was 0.48% of concentration in accordance of the minimum rate that has been tested in melon foliar applications to control mildews. The test plot receives drip irrigation in the same volume as control.

The procedure was as follows: (i) irrigating water (no additives) the first two hours; (ii) irrigating water containing 0.48% TTO formulation for 30 min; and (iii) irrigating water (no additives) volume to match control plot (total 6,600 liter for the test plot. Thirteen days later another sample was taken from the same test plot. Treatment protocol was repeated during 30 days post melon plantation, in accordance to the farm's irrigation protocol, with no signs of any phyto-toxiciy or negative effect on plants in the test plot.

TABLE 6
Disinfecting Effect of biocide composition containing 66% TTO at
0.48% emulsion on soil containing pathogenic Fungi and Nematode

| | | Pathogenic | | | | | |
|---|---|---|---|---|---|---|---|
| | Pathogenic | Fungi, | Meloidogyne, | | | | |
| Sample | bacteria | Fusariun spp. | Eggs | Larva | Aphelenchus, | Hoplolimus | Rotylenchulus. |
| Per | None | Yes | 200 | none | 150 | 125 | 300 |
| treatment | | | | | | | |
| Post | None | None | 125 | none | 25 | 100 | 125 |
| treatment | | | | | | | |

### EXAMPLE 14

The effect of a composition based on the above containing TTO (66%, weight percent) was tested against Cottonseed bug (Oxycarenus hyallinipennis), a cotton plant pathogenic Arachnid.

TABLE 7
An average measure of live Cottonseed bugs per plant over time
after treatment with disinfecting TTO-containing emulsion

| Plant | Day 0 | Day 4 | Day 8 |
|---|---|---|---|
| control | 6.7 | 7.2 | 4.9 |
| 0.25% | 8.7 | 5.5 | 6.0 |
| 0.5% | 7.0 | 5.2 | 3.8 |
| 1% | 7.4 | 3.8 | 1.9 |
| Commercial standard | 7.7 | 5.2 | 4.3 |

### EXAMPLE 15

The qualitative effect of a composition containing 16% TTO and an extract of Sophora japonica, was tested against the soil resident Aphid Eriosoma lanigerum. Good eradication in lab soil samples was obtained.

### EXAMPLE 16

The effect of a composition based on the soil-pathogen disinfectant comprising TTO (66%, weight percent) defined above, was tested against soil pathogens in a melon field which was treated in Example 2. Two treatments were applied after melon plants were transplanting. The first treatment was applied fifteen days after transplanting; while a second treatment was applied twenty five days after transplanting. In both cases, the 0.3 hectare field was irrigated by a single pump. The composition rate was 0.1% of concentration. The procedure was as follows: water was irrigated at the first 2 h, and then, in a period of 0.5 h, the TTO-based disinfectant was applied into 825 liters of irrigating water, to give a final concentration of 0.122%. Then, during another 1.5 h, the rest of irrigated water was applied.

Some 15 days after this second application, another soil/roots sample was taken from the same field (Sample # 1). Similar sample was soil/root sample was taken from a side field treated with Metam sodium (Sample # 2).

Laboratory analysis of soil/root samples of Sample # 1 found just a few mycelium without spores, being suspected as lab contamination. Moreover, some 550 nematode eggs from the gene Meloydogine were found.

Laboratory analysis of soil/root of Sample # 2 found growth of Fusarium in one out of the ten samples; Aspergillus in another sample out of the ten samples; and mycelium without spores. In both samples no root knot nematodes from the genes Hoplolimus, and Rotylenchulus were detected.

The experiment shows that TTO-based soil-pathogen disinfectants of the present invention have soil activity on controlling soil soil-borne pests detected especially Fusarium spp., and root knot nematodes from the genes Meloidogyne Aphelenchus, Hoplolimus, and Rotylenchulus.

The experiments show that post planting applications of the TTO-compositions of the invention are not phyto-toxic to melon plants at a rate of 0.122% or lower. Lab analysis indicates that the mycelium without spores found in both Samples is considered as a lab contaminant; due to the fact the roots were not showing harmed tissue. The experiments also show that TTO-based soil-pathogen disinfectants of the present invention have soil activity on controlling soil soil-borne pests and root knot nematodes.

TABLE 1
Disinfecting Effect of biocide composition containing 66% TTO at
0.48% emulsion on soil containing plant pathogenic organisms

| | | Pathogenic | | | | | |
|---|---|---|---|---|---|---|---|
| | Pathogenic | Fungi, | Meloidogyne, | | | | |
| Sample | bacteria | Fusariun spp. | Eggs | Larva | Aphelenchus, | Hoplolimus | Rotylenchulus. |
| Per | None | Yes | 200 | none | 150 | 125 | 300 |
| treatment | | | | | | | |
| Post | None | None | 125 | none | 25 | 100 | 125 |
| treatment | | | | | | | |

1. A biocompatible composition comprising TTO in a stable water-in-oil emulsion of alkali or ammonium salts of organic fatty acid wherein said emulsion is also stable when converted into an oil-in-water emulsion.

2. The biocompatible composition according to claim 1, useful for plant protection, excluding roots and seeds, from their pathogens, wherein said TTO-based emulsion is non-phytotoxic.

3. The biocompatible composition according to claim 1, wherein the organic fatty acids comprising linear or branched alkyl chains of 6<C<22.

4. The biocompatible composition according to claim 1, wherein at least a portion of the organic fatty acid is saturated fatty acid selected among lauric acid, myristic acid, palmitic acid, stearicacid, arahinoic acid, behenic acid, lignocerinic acid or any mixture thereof.

5. The biocompatible composition according to claim 1, wherein at least a portion of the organic fatty acid is unsaturated fatty acids selected among decenoic acid, dodecenoic acid, palamitinoleic acid, oleic acid, lonoleic acid, undecelenic acid, sorbic acid, recinoleic acid or any mixture thereof: and/or wherein at least a portion of the organic fatty acid is selected among tall oil acids, tall oil fatty acid (TOFA), naftenic acids, rosin acids and any mixture thereof.

6. The biocompatible composition according to claim 1, additionally comprising at least one etheric oil; said oil is especially selected for lavender (Lavandula angustifolia) oil, pine (Pinus sylvestris) oil, manuka (Leptospermum scoparium) oil, kanuca (Kunzea ericoides) oil, eucalyptus (Eucalyptus globulus) oil, bergamot (Citrus bergamia) oil, clove (Eugenia caryaphylata) oil, lemon (Citrus limoneum) oil, lemon grass (Cymbpogon citratus) oil, rosemary (Rosmarinus officialis) oil, geranium (Pelargonium graveoleus) oil, mint oil, or any mixture thereof.

7. A water-in-oil emulsion according to claim 1, wherein the concentration of said TTO is about 10% to about 70% (weight percent), based on the total weight of said emulsion, and, when converted into an oil-in-water emulsion the concentration of said TTO is about 0.1% to about 1.5% (weight percent), based on the total weight of said emulsion.

8. A water-in-oil emulsion according to claim 1, wherein the concentration of said TTO is about 10% to about 70% (weight percent), based on the total weight of said emulsion, and, when converted into an oil-in-water emulsion the concentration of said TTO is about 0.00015% to about 1.5% (weight percent), based on the total weight of said emulsion.

9. The biocompatible composition according to claim 6, wherein the concentration of the etheric oil (admixed to the emulsion) is between 0.01% by weight to 50% by weight.

10. The biocompatible composition according to claim 1, wherein the terpinen-4-ol concentration in the TTO is in the range of 20% by weight to 50% by weight.

11. The biocompatible composition according to claim 1, wherein the alkali and ammonium salts comprising at least one compound selected from sodium, potassium and/or ammonium hydroxides, carbonates, bicarbonates or any mixture thereof.

12. The biocompatible composition according to claim 1, wherein said emulsion comprising surfactants selected from a group consisting of cationic, anionic or zwitterionic, non-ionic or a mixture thereof.

13. The biocompatible composition according to claim 1, additionally comprising antioxidants, especially tocopherol, retinol, ascorbates or any mixture thereof.

14. The biocompatible composition according to claim 1, characterized by a non-phytotoxic broad spectrum disinfectant or repellant activity selected from kanacidic, insectecidic, archanidacide, antibiotic, fungicidic, nematocidic, bacteriocidic activities or any mixture thereof.

15. The biocompatible composition according to claim 1, additionally comprising tetracyclo-quinolizindine alkaloids, natural pyrethrines and products of chrysanthemum Daisy, Tanacetum cinerariarfolium, matrine, oxymatrine or any mixture thereof; and especially wherein the alkaloids are extracted from the group consisting of chrysanthemum Daisy, Tanacetum cinerariarfolium, Sophora japonica, Sophora subprostrata, Sophora alopecuroides or any combination thereof.

16. The biocompatible composition according to claim 1, wherein said TTO comprising at least one oil selected from a group consisting of terpinen-4-ol, a-terpineol, 1,8-cineole, α and γ terpinen or a combination thereof.

17. A biocompatible ozone-safe biocides and repellants comprising soil-treating disinfectants according to claim 1.

18. A non-phytotoxic soil-treating disinfectant according to claim 17, especially targeting plant pathogens; especially bacteria, nematodes and/or fungi.

19. The biocompatible composition according to claim 1, useful for the management of fungi pathogens in aquaculture, especially in fish farming, and particularly in treating Saprolegnia infections.

20. A method of producing a biocompatible biocide composition; said method comprising:
(a) admixing water-immiscible ingredients, optionally, admixing up to 100% of TTO;
(b) admixing water-miscible ingredients, especially alkali or ammonium salts such that a stable water-in-oil emulsion is obtained; and,
(c) optionally, admixing the remaining TTO in said emulsion.

21. The method according to claim 20, comprising:
a. producing a water-in-oil emulsion; the concentration of said tea tree oil is about 15% to about 70% (weight percent), based on the total weight of said emulsion; and,
b. converting said water-in-oil emulsion into an oil-in-water emulsion, the concentration of said tea tree oil is about 0.1% to about 1.5% (weight percent), based on the total weight of said emulsion.

22. The method according to claim 20, useful for the production of non-phytotoxic TTO-based plant-protecting emulsion.

23. The method according to claim 20, wherein said water-immiscible ingredients comprising organic fatty acids selected from a group consisting of linear or branched alkyl chains of 6<C<22; especially wherein at least a portion of the organic fatty acid is saturated fatty acid selected among lauric acid, myristic acid, palmitic acid, stearic acid, arahinoic acid, behenic acid, lignocerinic acid or any mixture thereof; especially wherein at least a portion of the organic fatty acid is unsaturated fatty acids selected among decenoic acid, dodecenoic acid, palamitinoleic acid, oleic acid, lonoleic acid, undecelenic acid, sorbic acid, recinoleic acid or any mixture thereof; especially wherein at least a portion of the organic fatty acid is selected among tall oil acids, tall oil fatty acid, naftenic acids, rosin acids and any mixture thereof.

24. The method according to claim 20 additionally comprising admixing water-immiscible ingredients comprising at least one etheric oil; especially wherein the etheric oil is selected for a group consisiting of lavender (Lavandula angustifolia) oil, pine (Pinus sylvestris) oil, manuka (Leptospermum scoparium) oil, kanuca (Kunzea ericoides) oil, eucalyptus (Eucalyptus globulus) oil, bergamot (Citrus bergamia) oil, clove (Eugenia caryaphylata) oil, lemon (Citrus limoneum) oil, lemon grass (Cymbpogon citratus) oil, rosemary (Rosmarinus officialis) oil, geranium (Pelargonium graveoleus) oil, mint oil or any mixture thereof; especially wherein the total concentration of said at lest one etheric oil (admixed to the emulsion) is between 0.01% by weight to 50% by weight or especially wherein the terpinen-4-ol concentration in the tea tree oil is in the range of 20% by weight to 50% by weight.

25. The method according to claim 20, additionally comprising step or steps of admixing antioxidants and/or surfactants; especially wherein said surfactants are selected from a group consisting of cationic, anionic or zwitterionic, non-ionic or a mixture thereof; especially wherein said antioxidants, are selected from a group consisting of tocopherol, retinol, ascorbates or any mixture thereof.

26. The method according to claim 20, wherein the emulsion is characterized by a non-phytotoxic broad spectrum disinfective and/or insect repellant activities selected, especially wherein said disinfective activity is selected from a group of activities consisting of kanacidic, insectecidic, arachnidacidic antibiotic, fungicidic, nematocidic, bacteriocidic activities or any combination thereof.
